(19) United States
(12) Patent Application Publication
(10) Pub. No.: US 2004/0229825 A1
(43) Pub. Date: Nov. 18, 2004

(54) Pharmaceutical composition for treatment of infection with drug resistant bacterium and disinfectant

(76) Inventors: Tomihiko Higuchi, Tokushima-ken (JP); Hirofumi Shibata, Tokushima-ken (JP); Takayuki Nakada, Gifu-ken (JP); Yoichi Sato, Gifu-ken (JP)

Correspondence Address: ERIK S. SPECTOR, BACON & THOMAS PLLC, 625 Slaters Lane Fourth floor, Alexandria, VA, 22314, US

(21) Appl. No.: 10/436,150

(22) Filed: May 13, 2003

(51) Int. Cl. A61K 31/7048; A61K 31/353

(52) U.S. Cl. 514/27; 514/456

(57) ABSTRACT

The present invention provides a pharmaceutical composition or a method for treatment of infections with a drug resistant bacterium including a flavonoid as an active ingredient, and also, a pharmaceutical composition or a method for treatment of infections with a drug resistant bacterium and a disinfectant including a flavonoid which can enhance efficacy of a β-lactam antibiotic, and said β-lactam antibiotic.

### BACKGROUND OF THE INVENTION

The present invention relates to a pharmaceutical composition for treatment of infections with a drug resistant bacterium comprising a flavonoid or a derivative thereof as an active ingredient, and a pharmaceutical composition for treatment of infections with a drug resistant bacterium using activity of a flavonoid which enhances efficacy of β-lactam antibiotics, as well as disinfectants exhibiting an antibacterial activity on drug resistant bacteria, which comprises a flavonoid as an active ingredient.

Penicillin, which is the first antibiotic, has a β-lactam ring, and has exerted an excellent efficacy toward Staphylococci. However, penicillin resistant bacteria which produce an enzyme i.e., penicillinase (β-lactamase), that degrades penicillin, emerged. In regard to these penicillin resistant bacteria, almost all problems appeared to be solved in clinical aspects by research and development of penicillinase resistant penicillin such as methicillin and cephems antibiotics, however, MRSA emerged to which all of the β-lactam agents are ineffective. In other words, MRSA is multiple drug resistant Staphylococcus aureus having broad resistance to not only penicillin antibiotics but also cephem antibiotics and aminoglycoside antibiotics. In recent years, as a result of abuse of third-generation cephem antibiotics which have weak antibacterial potency on staphylococci, bacteria which are resistant to these antibiotics have selectively proliferated. Such bacteria have come to spread in a hospital, which have resulted in critical social problems as principle causative bacteria of hospital acquired infection. Examples of currently used antibiotics for MRSA infections include vancomycin (VCM) and the like, however, short term bactericidal action of VCM is anything but potent, and VCM is involved in problems of serious side effects such as auricular toxicity and renal toxicity. In addition, combinations of multiple antibiotics have been conventionally investigated aiming at the enhancement of antibacterial potency. For example, a combination of an aminoglycoside agent with a β-lactam agent, phosphomycin with a β-lactam agent, and the like has been attempted, however, the effects by such combination are not necessarily satisfactory. There exist urgent needs to the development of novel antibacterial drugs which are effective on such resistant bacteria.

The inventors found an interesting fact during the search for compounds having anti-MRSA activities among Chinese herbal medicine with no or weak side effects, that various types of flavonoids suppress the resistance against β-lactam agents, and induce the sensitivity. The present invention was accomplished on the basis of such findings. It has not been reported that a flavonoid has antibacterial activities on resistant bacteria, and moreover, enhancing activities on the resistant bacteria by combinations of a certain type of β-lactam antibiotics with a flavonoid have been also unknown.

### SUMMARY OF THE INVENTION

The first aspect of the invention is a pharmaceutical composition for treatment of an infection with a drug resistant bacterium which comprises a flavonoid as an active ingredient.

The second aspect of the invention is a pharmaceutical composition for treatment of an infection with a drug resistant bacterium comprising a flavonoid which can enhance efficacy of a β-lactam antibiotic, and said β-lactam antibiotic, using an activity of a flavonoid which can enhance the efficacy of the β-lactam antibiotic on the drug resistant bacteria.

The third aspect of the invention is a disinfectant for drug resistant bacteria comprising a flavonoid, which can enhance efficacy of a β-lactam antibiotic, and said antibiotic.

Many of flavonoids are insoluble in water, and for example, flavone is insoluble in water at ambient temperature. It has been found that a flavonoid can be facilitated to dissolve into water by adding an amino acid. Accordingly, the invention provides a pharmaceutical composition which further comprises an amino acid as a solution adjuvant for flavonoids.

According to the invention, by the use of the flavonoid of the present invention in combination with an antibiotic or an antibacterial drug, the efficacy of β-lactam antibiotics on the resistant bacteria can be enhanced. An amount of β-lactam antibiotic to be used can be reduced and an opportunity for bacteria to acquire the resistance against antibiotics can be decreased in advance.

### DETAILED DESCRIPTION OF THE PREFERRED EMBODIMENTS

A flavonoid includes a flavone as a mother compound, which is a ketone compound having a ring condensed with a benzene and a pyran groups as a skeleton, and a phenyl group as a side group. The flavonoid has usually several substituents and may also be an analogous compound in which a pyron ring is hydrogenated or opened. Many of them are known as plant pigments. The flavonoids of the invention may be said flavonoid or derivatives thereof, or mixtures of two or more of the flavonoids.

In addition, the flavonoids and β-lactam antibiotic of the invention may include any of the pharmaceutically acceptable salts. Pharmaceutically acceptable salts refer to salts acceptable in pharmaceutical industry, for example, salts of sodium, potassium, calcium and the like, amine salts of procaine, dibenzylamine and the like, and acid addition salts such as hydrochlorides and the like.

Examples of drug resistant bacteria include methicillin resistant Staphylococcus aureus (MRSA), penicillinase producing Staphylococcus aureus, vancomycin resistance enterococci (VRE), vancomycin resistance Staphylococcus aureus (VRSA), penicillin resistance Streptococcus pneumoniae (PRSP), substrate specificity expanded β-lactamase (ESBLSs), and the like, preferably MRSA, and may be penicillinase producing Staphylococcus aureus.

Examples of the flavonoid of the invention having an antibacterial activity on drug resistant bacteria include flavones such as 6,7-dihydroxyflavone, 7,8-dihydroxyflavone, 7,4′-dihydroxyflavone, 3′,4′-dihydroxyflavone, and derivatives thereof or mixtures thereof; flavonols such as fisetin, kaempferid, morin, myricetin, and derivatives thereof or mixtures thereof; flavanones such as liquiritigenin, naringenin, and derivatives thereof or mixtures thereof; flavanonols such as dihydrorobinetin, fustin which, and derivatives thereof or mixtures thereof; anthocyanidins such as cyanidin, pelargonidin, and derivatives thereof or mixtures thereof; and chalcones such as phloretin, butein, and derivatives thereof or mixtures thereof.

Preferably, examples of the flavonoid of the invention are 6,7-dihydroxyflavone, 7,8-dihydroxyflavone, 3′,4′-dihydroxyflavone, fisetin and kaempferid.

Examples of flavonoids used for enhancing efficacy of β-lactam antibiotic include flavones such as flavone, apigenin, luteolin, 6,7-dihydroxyflavone, 7,8-dihydroxyflavone, 3′,4′-dihydroxyflavone; flavanonols such as rutin, kaempferol; flavanonols such as (+)-taxifolin; flavan-3-ols such as (−)-gallocatechin; and chalcones such as chalcone, and the derivatives thereof or the mixtures of the same. For brevity's sake, flavonoids and derivatives thereof specifically described above may be referred to as “a flavonoid” or “flavonoids” hereinafter in the specification.

Although the activity of rutin could not be determined in an in vitro test for antibacterial activities (data not shown), excellent effects could be determined in vivo by the use of rutin in combination with an antibiotic (see, Pharmacological Experiment 5). Rutin can be used in any forms including water-soluble rutin, sugar-transferred rutin, clathrated rutin and the like.

Examples of the β-lactam antibiotics of the invention include benzylpenicillin, phenoxymethylpenicillin, phenethicillin, propicllin, ampicillin, methicillin, oxacillin, cloxacillin, flucloxacillin, dicloxacillin, hetacillin, talampicillin, bacampicillin, lenampicillin, amoxicillin, ciclacillin, carbenicillin, sulbenicillin, ticarcillin, carindacillin, carfecillin, piperacillin, mezlocillin, aspoxicillin, cephaloridine, cefazolin, cefapirin, cephacetrile, ceftezole, cephaloglycin, cephalexin, cephalexin, cefatrizine, cefaclor, cefroxadine, cefadroxil, cefamandole, cefotiam, cephalothin, cephradine, cefuroxime, cefoxitin, cefotaxime, ceftizoxime, cefinenoxime, cefodizime, ceftriaxone, cefuzonam, ceftazidime, cefepim, cefpirome, cefozopran, cefoselis, ceflurenam, cefoperazone, cefpimizole, cefpiramide, cefixime, cefteram pivoxil, cefpodoxime proxetil, ceftibuten, cefetamet pivoxil, cefdinir, cefditoren pivoxil, cefcapene pivoxil, cefsulodin, cefoxitin, cefinetazole, latamoxef, cefotetan, cefbuperazone, cefminox, flomoxef, aztreonam, carumonam, imipenem, panipenem, meropenem, viapenem, faropenem, ritipenem acoxil, or mixtures thereof, preferably, benzylpenicillin, phenethicillin, methicillin, oxacillin, carbenicillin, cefapirin, cefradine, cefuroxime, cefoxitin, cefotaxime, and panipenem and mixtures thereof.

The antibiotics may be in the form of a pharmaceutically acceptable salt. Pharmaceutically acceptable salts refer to salts which can be generally used as salts of an antibiotic in pharmaceutical industry, including for example, salts of sodium, potassium, calcium and the like, and amine salts of procaine, dibenzylamine, ethylenediamine, ethanolamine, methylglucamine, taurine, and the like, as well as acid addition salts such as hydrochlorides, and basic amino acids and the like.

The flavonoids and derivative thereof of the invention can be administered parenterally, orally or topically, like in the case of conventional antibiotics. In general, they can be advantageously administered in the form of injection, which is prepared by the conventional process. The injection includes such a form, e.g., freeze-dried of injection that is dissolved in a suitable vehicle, e.g., sterilized distilled water, saline and the like before using.

Moreover, a flavonoid can be orally administered in combination of a β-lactam antibiotic in various type of formulations for oral administration. Examples of the formulation include tablet, capsule, sugarcoated tablet and the like, liquid solution or suspension.

For prophylaxis and/or therapy, total dose of both components, a flavonoid and a β-lactam antibiotic, may depend on said components to be used, the ratio thereof, the age, body weight, symptoms of the patient and the route for administration. For example, when administered to an adult (body weight: about 50 kg), 10 mg-2 g in total weight of both components to be used per single dosage is administered from once to three times per day. The dose and route for administration are selected in order to achieve the best therapeutic effects.

According to the invention, the weight ratio of both components in combination or admixed together can be in a wide range. In addition, since the combination ratio of both components depends on a type of infection, severity of the patient to be treated and the antibiotic to be used in combination, it is not particularly limited. Accordingly, the concentration of both components having an expectative effect can be achieved in the range of usual dosage.

The pharmaceutical composition is usually prepared according to the conventional process, and is administered in a pharmaceutically suitable form. For example, solid form for oral use may be formulated by the combination of active compounds with a diluent such as lactose, dextrose, saccharose, cellulose, and cornstarch and potato starch, a lubricant such as silica, talc, stearic acid, magnesium stearate or calcium stearate, and/or polyethylene glycol, a binder such as starch, gum Arabic, gelatin, methyl cellulose, carboxymethylcellulose, polyvinyl pyrrolidine, a disintegrant such as starch, alginic acid, alginate, glycolic acid starch sodium, a foaming agent, a colorant, a sweetening agent, a wetting agent such as lecithin, polysorbate, lauryl sulfate, and the like and pharmaceutically inactive and nontoxic substances well known in the art.

The above-described pharmaceutical preparation may be manufactured in a manner that is known in the art, e.g., by means of conventional mixing, granulating, tabletting, and coating, if desired, sugar coating processes.

In case of parenteral administration, suppository for rectal application or injection may be used, preferably, injection. The injection may be formulated in the form of aqueous solutions, solutions dissolved before using, and suspensions. Although the forms are different in appearance, they are substantially identical in respect of requiring sterilization of the active ingredient by an appropriate method, followed by directly placing into a vessel, and sealing.

Most convenient formulation process includes a process in which the active ingredient is sterilized by an appropriate method, thereafter separately, or after being physically mixed, the aliquot thereof is divided into plural dosage formulations. For a liquid dosage form, an active ingredient is dissolved in an appropriate medium and the resulting solution is sterilized and filtrated followed by filling in an appropriate ampoule or vial, and sealing. In this case, the appropriate media is usually distilled water for injection, but is not limited thereto in accordance with the invention. Aqueous injection may contain additives such as soothing agents which have local anesthetic effect, such as procaine hydrochloride, xylocaine hydrochloride, benzyl alcohol and phenol, antiseptic agents such as benzyl alcohol, phenol, methyl or propylparaben and chlorobutanol, buffering agents such as a sodium salt of citric acid, phosphoric acid, acetic acid, solution adjuvants such as ethanol, propylene glycol, arginine hydrochloride, stabilizing agents such as L-cysteine, L-methionine, L-histidine, and tonicity agents, if required.

The flavonoids of the invention can be formulated as external preparations having an antibacterial action on drug resistant bacteria. The flavonoids of the invention can be formulated as an antibacterial agent or a bactericidal agent by mixing with ≢-lactam antibiotics. These antibacterial agents or bactericidal agents are used at a concentration of 0.1-10% (by weight or volume) to disinfect instruments such as scissors, scalpels, catheters, as well as excrements of patients, and to irrigate skins, mucosa and wounds.

Pharmacological Experiment 1

Antibacterial Activity of Flavonoids on MRSA

MRSA were employed as bacteria to be tested. The antibacterial activity was determined according to the agar plate dilution method defined by Japan Society of Chemotherapy (Chemotherapy 29(1), 76-79(1981)). The employed plate medium for the measurement of sensitivity was semisynthetic medium based on Mueller-Hinton Agar, and the bacteria solution for seeding was prepared by incubating the test bacterium in Mueller-Hinton Broth at 37° C. for 20 hours, followed by diluting in 0.85% saline to give 10⁶ CFU/mL. The test samples were produced in two times-serial dilution method. To this plate medium for the measurement of sensitivity, was seeded a bacteria solution with a Micro Planter® (Sakuma Seisakusho). After incubating at 37° C. for 20 hours, a minimum inhibitory concentration (MIC) was determined. MIC value is defined as a minimum concentration at which the growth of the bacteria was completely inhibited. In addition, MIC₅₀ indicates a concentration yielding the proliferation inhibitory effect in 50% of total number of MRSA strains, whilst MIC₉₀ indicates a concentration yielding the proliferation inhibitory effect in 90% of total number of MRSA strains.

Antibacterial activities on MRSA 20 strain by flavonoids are shown in Table 1.

TABLE 1

MIC
(μg/mL)

| compound | range | MIC₅₀ | MIC₉₀ |
|---|---|---|---|
| flavone | 31.3->250 | 62.5 | 125 |
| 6,7-dihydroxyflavone | 31.3->250 | 62.5 | 125 |
| 7,8-dihydroxyflavone | 31.3-125 | 62.5 | 125 |
| 7,4′-dihydroxyflavone | 125-500 | 250 | 500 |
| 3′,4′-dihydroxyflavone | 62.5-250 | 125 | 250 |
| fisetin | 62.5-125 | 125 | 125 |
| kaempferid | 15.6-125 | 125 | 125 |
| morin | 250->250 | 500 | >500 |
| myricetin | 250-500 | 500 | 500 |
| liquiritigenin | 250-500 | 500 | 500 |
| naringenin | 500->500 | 500 | >500 |
| dihydrorobinetin | 250 | 250 | 250 |
| fustin | 250->500 | 500 | 500 |
| cyanidin | 250->500 | 250 | >500 |
| palagonidin | 250 | 250 | 250 |
| butein | 125-250 | 250 | 250 |
| phloretin | 125-250 | 250 | 250 |

Results

As shown in Table 1, antibacterial activities on MRSA were demonstrated with a flavonoid alone. In particular, 6,7-dihydroxyflavone, 7,8-dihydroxyflavone, 3′,4′-dihydroxyflavone, fisetin, and kaempferid demonstrated potent antibacterial activity.

Pharmacological Experiment 2

Enhancement of the Antibacterial Activity of Methicillin by Various Flavonoids on MRSA

Antibacterial activities of methicillin on MRSA when 50 μg/mL of flavonoids were added are shown in Table 2.

Antibacterial Activities of Methicillin on MRSA with 50 μg/mL of Various Flavonoids

TABLE 2

| | MIC(μg/ml) of |
|---|---|
| compound | methicillin |
| methicillin | 1024 |
| flavone | 2 |
| apigenin | 1 |
| kaempferol | 2 |
| luteolin | 1 |
| 6,7-dihydroxyflavone | <2 |
| 7,8-dihydroxyflavone | <2 |
| 3′,4′-dihydroxyflavone | <2 |
| (+)-taxifolin | 2 |
| (−)-gallocatechin | 2 |
| chalcone | 2 |

Results

As shown in Table 2, although MIC for methicillin alone was 1024 μg/mL, sensitivity was revealed to be elevated to 2 μg/mL or less by adding various types of flavonoids at 50 μg/mL.

Pharmacological Experiment 3

Enhancement of the Antibacterial Activity of Various Antibiotics by Flavone on MRSA

Antibacterial activities of various antibiotics on MRSA when 50 μg/mL of flavone was added are shown in Table 3.

Antibacterial Activities of Various β-Lactam Antibiotics on MRSA with 50 μg/mL of Flavone

TABLE 3

MIC (μg/mL)

| antibacterial agent | −flavone | +flavone |
|---|---|---|
| benzylpenicillin | 64 | 32 |
| phenethicillin | 128 | 16 |
| methicillin | 1024 | 4 |
| oxacillin | 512 | 1 |
| carbenicillin | >256 | 4 |
| cefapirin | 128 | <0.016 |
| cefradine | >256 | 1 |
| cefuroxime | 1024 | 512 |
| cefoxitin | 512 | 32 |
| cefotaxime | >1024 | 64 |
| panipenem | 64 | 0.002 |

−flavone: antibiotics alone
+flavone: addition of 50 μg/mL of flavone

Results

As shown in Table 3, it was found that by adding flavone at 50 μg/mL, the antibacterial activities of β-lactam antibiotics were enhanced.

Pharmacological Experiment 4

Enhancement of the Antibacterial Activity of Benzylpenicillin by Flavone on Penicillinase Producing Staphylococcus aureus.

The effects of flavone in combination with benzylpenicillin or methicillin on penicillinase producing Staphylococcus aureus are shown in Table 4.

Effects of Flavone in Combination with Benzylpenicillin or Methicillin on Penicillinase Producing Staphylococcus aureus.

TABLE 4

| | MIC (μg/mL) |
|---|---|
| benzylpenicillin | 1.56 |
| benzylpenicillin + flavone 50 μg/mL | 0.39 |
| methicillin | 0.98 |
| methicillin + flavone 50 μg/mL | 0.98 |

Results

As shown in Table 4, although MIC for benzylpenicillin alone which is degraded by penicillinase was 1.56 μg/mL, the antibacterial activity was enhanced up to 0.39 μg/mL by adding flavone at 50 μg/mL. Moreover, as for methicillin which is not originally degraded by penicillinase, the antibacterial activity was not altered in the presence or absence of flavone.

Pharmacological Experiment 5

Antibacterial Activity on MRSA Infected Mouse

MRSA was incubated with Brain-Heart Infusion medium at 37° C. for 18 hours, and then the bacteria were collected and washed, followed by resuspension in saline. The suspension was admixed in an aqueous 5% gastric mucin solution and the concentration was adjusted to a predetermined value. Then, 0.2 mL of the mixture was intraperitoneally administered to an ICR strain SPF mouse. At one hour after the infection, various concentrations of β-lactam agents and rutin (0.2 mL) was subcutaneously administered, and survival rate was analyzed after 5 days and thus the effectiveness was evaluated. In addition, the effectiveness by oral administration (0.2 mL) was evaluated and various concentrations of β-lactam agents and rutin (0.2 mL) were administered using a gastric probe 1 hour or 5 hours prior to the infection.

The effects of rutin in combination with a β-lactam agent on MRSA infected mouse are shown in Tables 5 to 12.

Effects of Oxacillin Alone on MRSA Infected Mouse

TABLE 5

| | |
|---|---|
| antibiotics | oxacillin |

| | | | | | |
|---|---|---|---|---|---|
| concentration | 0.5 | 1 | 5 | 10 | 15 |
| (mg/mouse) | | | | | |
| survival | 20 | 0 | 20 | 20 | 0 |
| rate (%) | | | | | |

Effects of Cephapirin Alone on MRSA Infected Mouse

TABLE 6

| | |
|---|---|
| antibiotics | cephapirin |

| | | | | | |
|---|---|---|---|---|---|
| concentration | 0.5 | 1 | 5 | 10 | 15 |
| (mg/mouse) | | | | | |
| survival | 20 | 20 | 20 | 20 | 40 |
| rate (%) | | | | | |

Effects of Combination of Oxacillin or Cephapirin with Rutin on MRSA Infected Mouse (Subcutaneous Administration)

a) Rutin Alone

TABLE 7

| | |
|---|---|
| flavonoid | rutin |

| | | | | |
|---|---|---|---|---|
| concentration | 1 | 5 | 10 | 15 |
| (mg/mouse) | | | | |
| survival | 40 | 40 | 20 | 40 |
| rate (%) | | | | |

b) Combination of Oxacillin or Cephapirin with Rutin (5 mg/mouse)

TABLE 8

| | | |
|---|---|---|
| antibiotics | oxacillin | cephapirin |

| | | | | |
|---|---|---|---|---|
| concentration | 10 | 15 | 10 | 15 |
| (mg/mouse) | | | | |
| survival | 40 | 60 | 0 | 80 |
| rate (%) | | | | |

c) Combination of Oxacillin or Cephapirin with Rutin (10 mg/mouse)

TABLE 8

| | | |
|---|---|---|
| antibiotics | oxacillin | cephapirin |

| | | | | |
|---|---|---|---|---|
| concentration | 10 | 15 | 10 | 15 |
| (mg/mouse) | | | | |
| survival | 80 | 60 | 20 | 100 |
| rate (%)L | | | | |

Effects in Combination of Oxacillin or Cephapirin with Rutin on MRSA Infected Mouse (Oral Administration)

a) Rutin Alone

TABLE 10

| | |
|---|---|
| flavonoid | rutin |

| | | | | |
|---|---|---|---|---|
| concentration | 1 | 5 | 10 | 15 |
| (mg/mouse) | | | | |
| concentration | 1 | 5 | 10 | 15 |
| (mg/mouse) | | | | |
| survival | 20 | 20 | 20 | 40 |
| rate (%) | | | | |

b) Combination of Oxacillin or Cephapirin with Rutin (5 mg/mouse)

TABLE 11

| | | |
|---|---|---|
| antibiotics | oxacillin | cephapirin |

| | | | | |
|---|---|---|---|---|
| concentration | 10 | 15 | 10 | 15 |
| (mg/mouse) | | | | |
| survival | 20 | 40 | 40 | 60 |
| rate (%) | | | | |

c) Combination of Oxacillin or Cephapirin with Rutin (10 mg/mouse)

TABLE 11

| | | |
|---|---|---|
| antibiotics | oxacillin | cephapirin |

| | | | | |
|---|---|---|---|---|
| concentration | 10 | 15 | 10 | 15 |
| (mg/mouse) | | | | |
| survival | 20 | 40 | 60 | 80 |
| rate (%) | | | | |

Results

As shown in Tables 5 to 12, although the administration of oxacillin alone to MRSA infected mouse resulted in the survival rate of 20% or less, subcutaneous administration of rutin (5, 10 mg/mouse) led to marked improvement of the survival rate. In particular, cephapirin (15 mg/mouse) in combination with rutin (10 mg/mouse) resulted in the survival rate of 100%. In like manner, oral administration in combination with rutin resulted in marked improvement of survival rate.

### TEST EXAMPLE 1

Auxiliary Effects on Dissolution of Rutin by L-Arginine Hydrochloride and L-Cysteine

When rutin was dissolved in water at an ambient temperature, it was insoluble in water (0 mg/mL). However, when rutin is dissolved into a solution containing 4 mmol of L-arginine hydrochloride, 1 mmol of L-cysteine and 8 mmol of 1 N NaOH, and then, the resulting solution is adjusted pH 8.5 with 1 N HCl followed by removing the precipitate, rutin exhibited the solubility of 49.8 mg/mL (see, Example 3).

### EXAMPLES

### Example 1

### Tablet

According to the conventional process, 50 mg of rutin, 1 g of lactose, 30.0 mg of starch, 50 mg of methylcellulose and 30 mg of talc were mixed to make ten tablets, which were then coated with sucrose.

### Example 2

### Injection

A sterile mixture containing 500 mg of rutin was placed in a sterilized vial which was then sealed. Before using, this mixture is dissolved in saline to give an injection.

EXAMPLE 3
(Injection)
Solution A

| | |
|---|---|
| L-arginine hydrochloride | 840 mg |
| L-cysteine | 121 mg |
| 1N NaOH | 8 mL |
| distilled water | q.s. |
| Total | 10 mL |

Solution B

| | |
|---|---|
| rutin | 664 mg |
| distilled water | q.s. |
| Total | 10 mL |

To the solution B, is added 3.5 mL of the solution A. After adjusting the pH of the mixture to 8.5 with HCl, the mixture is filtered to give an injectable.

### Example 4

### Disinfectant

Five g of flavone and 5 g of cephapirin are dissolved into 1000 mL of ordinary water to be used as a disinfectant.

1-19 (canceled).

20. A pharmaceutical composition for the therapy of an infection with a drug resistant bacterium, comprising a flavone selected from the group consisting of 6,7-dihydroxyflavone, 7,8-dihydroxyflavone, 3′4′-dihydroxyflavone and derivatives thereof, which can enhance efficacy of a β-lactam antibiotic, and said β-lactam antibiotic, as active ingredients.

21. The pharmaceutical composition according to claim 20 wherein said β-lactam antibiotic is selected from the group consisting of benzylpenicillin, phenethicillin, methicillin, oxacillin, carbenicillin, cefapirin, cephradine, cefuroxime, cefoxitin, cefotaxime, panipenem and mixtures thereof.

22. The pharmaceutical composition according to claim 20 or claim 21 wherein said drug resistant bacterium is MRSA.

23. A pharmaceutical composition in a form for systemic administration for the therapy of an infection with a drug resistant bacterium, comprising rutin or a derivative thereof, which can enhance efficacy of a β-lactam antibiotic, and said β-lactam antibiotic, as active ingredients.

24. The pharmaceutical composition according to claim 23 which is formulated for oral administration.

25. The pharmaceutical composition according to claim 23 which is formulated for injection.

26. The pharmaceutical composition according to claim 23 or claim 24 or claim 25 wherein said rutin is in the form of water-soluble rutin, sugar-transferred rutin or clathrated rutin.

27. The pharmaceutical composition according to claim 23 or claim 24 or claim 25 wherein said β-lactam antibiotic is selected from benzylpenicillin, phenethicillin, methicillin, oxacillin, carbenicillin, cefapirin, cephradine, cefuroxime, cefoxitin, cefotaxime, panipenem and mixtures thereof.

28. The pharmaceutical composition according to claim 23 or claim 24 or claim 25 wherein said drug resistant bacterium is MRSA.
